(19) United States
(12) Patent Application Publication
(10) Pub. No.: US 2020/0297279 A1
(43) Pub. Date: Sep. 24, 2020

(54) GARMENT SLEEVE PROVIDING BIOMETRIC MONITORING

(71) Applicant: CIPHER SKIN, Denver, CO (US)

(72) Inventors: Phillip Bogdanovich, Evergreen, CO (US); Craig Weller, Evergreen, CO (US); James Heather, Boston, MA (US)

(73) Assignee: CIPHER SKIN, Denver, CO (US)

(21) Appl. No.: 16/825,822

(22) Filed: Mar. 20, 2020

Related U.S. Application Data
(60) Provisional application No. 62/820,876, filed on Mar. 20, 2019.

(51) Int. Cl. A61B 5/00; A61B 5/11; A61B 5/024; A61B 5/053

(52) U.S. Cl. CPC A61B 5/6804; A61B 5/684; A61B 5/11; A61B 2562/0219; A61B 5/0533; A61B 5/0022; A61B 5/0024; A61B 5/024

(57) ABSTRACT

A sleeve that may be worn on a limb of a user is disclosed. The sleeve may be used to assess shape, position, and movement of the limb, as well as biometric properties of the user. The sleeve may include a geometric pattern of conductive elastic material interspersed with non-conductive elastic material. A plurality of sensor units may be integrated in the sleeve with portions of the conductive elastic material coupled between sensor units. The sensor units may assess resistances of the portions of the conductive elastic material between the sensor units. A positioning component may be positioned at a joint of the limb when the sleeve is worn. The sleeve may include inertial measurement units integrated in the sleeve and positioned on upper and lower portions of the joint. A processor may receive and assess data from the sensor units and the first and second inertial measurement units.

### RELATED APPLICATIONS

This application claims priority to U.S. Patent Application No. 62/820,876, filed on Mar. 20, 2019, which is incorporated by reference as if fully set forth herein. This application is related to U.S. patent application Ser. No. 16/049,114, filed on Jul. 30, 2018, which is incorporated by reference as if fully set forth herein.

### FIELD OF THE INVENTION

Embodiments disclosed herein relate to a garment sleeve. Certain embodiments disclosed herein relate to a garment sleeve for monitoring biometric information associated with a limb wearing the sleeve and associated methods.

### BACKGROUND

Wearable technology has become an increasingly more common resource for users to track and monitor their biometric data during physical activity and/or day-to-day activity. Devices such as wristbands, glasses, and watches have been developed that function to gather biometric data from an individual's body such as heart rate, force on a body, acceleration of a body, etc. These devices, however, may not be capable of tracking or generating a complex profile of a user's biometric data in combination with movement and body position of the user. Thus, there is still are needs for systems (e.g., a garments) that are capable of generating biometric data for real-time analysis and/or historical tracking of an individual's condition. Based on the foregoing currently existing technologies and processes for monitoring and analyzing biometric data may be enhanced and improved upon so as to provide increased functionality and quality of data for users. Such enhancements may facilitate a better understanding of biometric data, more accurate data, enhanced monitoring of biometric data, enhanced biometric data capture techniques, improved outputs generated based on the biometric data, among other benefits.

### SUMMARY

In certain embodiments, a system includes a fabric for being worn by a wearer. The fabric may include one or more layers. At least one layer of fabric may have a geometric pattern of conductive elastic material interspersed with non-conductive elastic material. A plurality of sensor units may be integrated in the fabric. At least a portion of the conductive elastic material may be coupled between at least two sensor units. The at least two sensor units may assess a resistance of the portion of the conductive elastic material between the at least two sensor units. A positioning component may be integrated in the fabric. The positioning component may be positioned at a joint of the limb when the fabric is worn on the limb. A first inertial measurement unit may be integrated in the fabric and positioned on a first side of the positioning component. A second inertial measurement unit may be integrated in the fabric and positioned on a second side of the positioning component. A processor may be integrated in the fabric. The processor may receive data from the sensor units and the first and second inertial measurement units. The processor may assess shape, position, and movement of the limb using the received data.

In certain embodiments, a method includes assessing a resistance of a portion of a conductive elastic material interspersed with non-conductive elastic material in a geometric pattern in a fabric. The resistance may be assessed using at least two sensor units coupled to the portion of the conductive elastic material. The fabric may be worn on a limb of a user. Motion of an upper portion of the limb may be assessed using a first inertial measurement unit integrated in a portion of the fabric worn on the upper portion of the limb. Motion of a lower portion of the limb may be assessed using a second inertial measurement unit integrated in a portion of the fabric worn on the lower portion of the limb. A processor integrated in the fabric may receive resistance data from the at least two sensor units and motion data from the first inertial measurement unit and the second inertial measurement unit. Shape, position, and movement of the limb of the wearer may be assessed using the received data.

In an embodiment, a system for monitoring biometric data by utilizing a garment sleeve is disclosed. The system may include a fabric for being worn on a limb of a wearer. In certain embodiments, the fabric comprises one or more layers. In certain embodiments, at least one layer of the one or more layers of the fabric may comprise a geometric pattern of conductive elastic material interspersed with non-conductive elastic material. The system may also include a plurality of nodes integrated in the fabric. A first portion of the conductive elastic material may be coupled between at least two nodes. The system may include a positioning component integrated in the fabric, a first inertial measurement unit integrated in the fabric and positioned on a first side of the positioning component, and a second inertial measurement unit integrated in the fabric and positioned on a second side of the positioning component. The system may further include a processor integrated in the fabric, which may be configured to assess a resistance of the first portion of the conductive elastic material between the at least two nodes and receive data from the first and second inertial measurement units.

### BRIEF DESCRIPTION OF THE DRAWINGS

Features and advantages of the methods and apparatus described herein will be more fully appreciated by reference to the following detailed description of presently preferred but nonetheless illustrative embodiments when taken in conjunction with the accompanying drawings in which:

FIG. 1 depicts a posterior view representation of an embodiment of a garment sleeve.

FIG. 2 depicts a lateral view representation of an embodiment of a garment sleeve.

FIG. 3 depicts a lateral cut-away representation of an embodiment of a sleeve.

FIG. 4 depicts an exploded side view of an embodiment of a multi-layer fabric panel.

FIG. 5 depicts a representation of an embodiment of a sensor unit.

FIG. 6 depicts a representation of an embodiment of a strain detection unit.

FIG. 7 depicts a representation of another embodiment of a strain detection unit.

FIG. 8 depicts a flowchart of an embodiment of a method of determining biometric properties of a user's limb using a sleeve.

FIG. 9 depicts a block diagram of one embodiment of an exemplary computer system.

FIG. 10 depicts a block diagram of one embodiment of a computer accessible storage medium.

FIG. 11 is a schematic diagram of a system that may be utilized to facilitate biometric monitoring by utilizing a garment sleeve according to an embodiment of the present disclosure.

FIG. 12 is a flow diagram illustrating a sample method for conducting biometric monitoring by utilizing a garment sleeve according to an embodiment of the present disclosure.

FIG. 13. is a schematic diagram of a machine in the form of a computer system within which a set of instructions, when executed, may cause the machine to perform any one or more of the methodologies or operations of the systems and methods for monitoring physical bodies by utilizing a flexible circuit design.

While the disclosure is susceptible to various modifications and alternative forms, specific embodiments thereof are shown by way of example in the drawings and will herein be described in detail. It should be understood, however, that the drawings and detailed description thereto are not intended to limit the disclosure to the particular form illustrated, but on the contrary, the intention is to cover all modifications, equivalents and alternatives falling within the spirit and scope of the present disclosure as defined by the appended claims. The headings used herein are for organizational purposes only and are not meant to be used to limit the scope of the description. As used throughout this application, the word “may” is used in a permissive sense (i.e., meaning having the potential to), rather than the mandatory sense (i.e., meaning must). Similarly, the words “include,” “including,” and “includes” mean including, but not limited to. Additionally, as used in this specification and the appended claims, the singular forms “a”, “an”, and “the” include singular and plural referents unless the content clearly dictates otherwise. The term “coupled” means directly or indirectly connected.

Various units, circuits, or other components may be described as “configured to” perform a task or tasks. In such contexts, “configured to” is a broad recitation of structure generally meaning “having structure that” performs the task or tasks during operation. As such, the unit/circuit/component can be configured to perform the task even when the unit/circuit/component is not currently on. Similarly, various units/circuits/components may be described as performing a task or tasks, for convenience in the description. Such descriptions should be interpreted as including the phrase “configured to.” Reciting a unit/circuit/component that is configured to perform one or more tasks is expressly intended not to invoke 35 U.S.C. § 112(f) interpretation for that unit/circuit/component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### DETAILED DESCRIPTION OF EMBODIMENTS

The following examples are included to demonstrate preferred embodiments. It should be appreciated by those of skill in the art that the techniques disclosed in the examples which follow represent techniques discovered by the inventor to function well in the practice of the disclosed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosed embodiments.

This specification includes references to “one embodiment” or “an embodiment.” The appearances of the phrases “in one embodiment” or “in an embodiment” do not necessarily refer to the same embodiment, although embodiments that include any combination of the features are generally contemplated, unless expressly disclaimed herein. Particular features, structures, or characteristics may be combined in any suitable manner consistent with this disclosure.

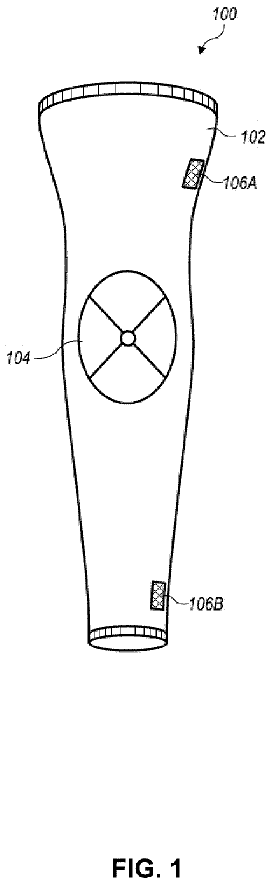

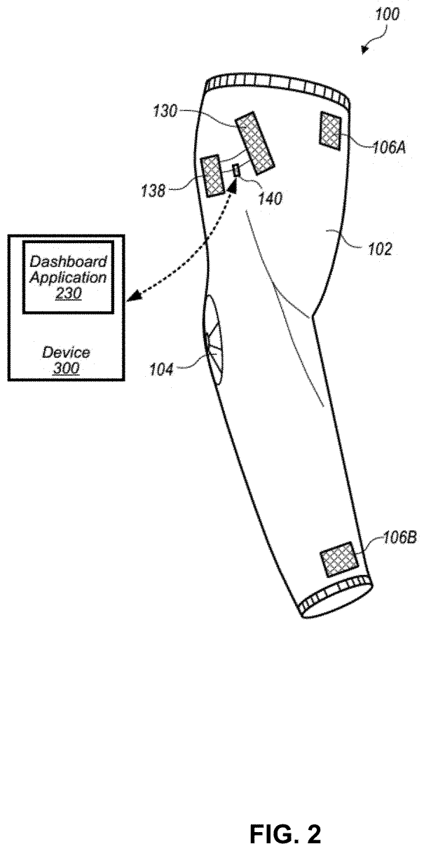

FIG. 1 depicts a posterior view representation of an embodiment of a garment sleeve 100. FIG. 2 depicts a lateral view representation of an embodiment of garment sleeve 100. Sleeve 100 may be, for example, a garment wearable by a user (e.g., a person or an animal) on a limb of the user (e.g., an arm or leg of the user). In certain embodiments, sleeve 100 is constructed of a quick-dry material with antistatic and anti-microbial properties. In some embodiments, sleeve 100 is form fitting (e.g., has an athletic fit) similar to a compression sleeve. For example, sleeve 100 may include form-fitting fabric that has an open interior defining an opening for the limb. Sleeve 100 may also include any form-fitting fabric with an open interior for any body part (e.g., neck, hand, foot, or torso).

In certain embodiments, sleeve 100 includes fabric 102. Sleeve 100 may, for example, be made up of fabric 102. In certain embodiments, fabric 102 may be included as part of sleeve 100 along with other components (e.g., other materials or structural components). In some embodiments, fabric 102 includes one or more fabric layers with elastic fibers similar to spandex. In some embodiments, fabric 102 includes a plurality of fabrics or yarns arranged in a woven and/or a knit pattern. For example, fabric 102 may include a plurality of materials interspersed together (e.g., interlaced or interwoven together). In some embodiments, fabric 102 includes fabric enhancements such as, but not limited to, improved moisture wicking, improved thermal management, and/or muscle group support.

Sleeve 100 may be made available in a number of sizes. For example, sleeve 100 may be available in in a range of sizes to accommodate different limb or body part diameters (e.g., different limb girths). Sleeve 100 may be available in different sizes to ensure the sleeve is form fitting and snug on the limb to accurately measure physiological responses. In some embodiments, sleeve 100 is designed to be worn for prolonged periods of time and/or under other equipment (e.g., under shirt sleeves or equipment pads).

In certain embodiments, sleeve 100 includes positioning component 104. In some embodiments, positioning component 104 may include a shaped component that conforms to a joint of the user's limb (e.g., the elbow or knee joint). The shaped component may be designed such that sleeve 100 is positioned at or near the same location in each instance that the sleeve is worn by the user. In some embodiments, positioning component 104 includes a marker or indicator. The marker or indicator may be used to mark or indicate proper position of sleeve 100. In some embodiments, positioning component 104 includes a combination of the shaped component and the marker or indicator. Using positioning component 104 to ensure sleeve 100 is properly positioned in repeated uses of the sleeve may provide more reliable and accurate results about shape, movement, and position of the limb using the sleeve, measured as described herein.

As shown in FIGS. 1 and 2, sleeve 100 may include one or more inertial measurement units 106. Inertial measurement units 106 may assess motion of the units to assess motion of portions of the limb inside sleeve 100. Inertial measurement units 106 may assess the physical position of limb in sleeve 100 in a three-dimensional space as well as complex motion of the limb and/or different portions of the limb. For example, inertial measurement units 106 may provide fast mapping (e.g., near real-time mapping) of complex motions including rotation, flexion, and extension of the limb or portions of the limb.

In some embodiments, inertial measurement units 106 provide accelerometer-based measurements. For example, inertial measurement units 106 may provide the position or orientation of sleeve 100 relative to perpendicular and/or relative to the ground. The position or orientation of sleeve 100 may provide the position or orientation of the limb relative to perpendicular and/or relative to the ground. Providing position and/or orientation of the limb (or portions of the limb) may allow an evaluator to determine the activity of the limb (e.g., throwing, running, swimming, etc.). In some embodiments, inertial measurement units 106 may provide position and/or orientation information relative to each other.

In certain embodiments, sleeve 100 includes inertial measurement unit 106A above the joint location of the sleeve (e.g., above positioning component 104) and inertial measurement unit 106B below the joint location. Inertial measurement units 106A, 106B may be capable of measuring motion with multiple degrees-of-freedom. For example, inertial measurement units 106A, 106B may measure motion with nine (9) degrees-of-freedom. Thus, sleeve 100 may be capable of measuring motion of the limb with nine degrees-of-freedom both above and below the joint location of the sleeve along with relative motion between portions of the limb above and below the joint location. Inertial measurement units 106 may provide data that may be used in combination with other data described herein (e.g., muscle motion/shape data, heart rate data, etc.) to provide a three-dimensional image of position and movement of the limb in space.

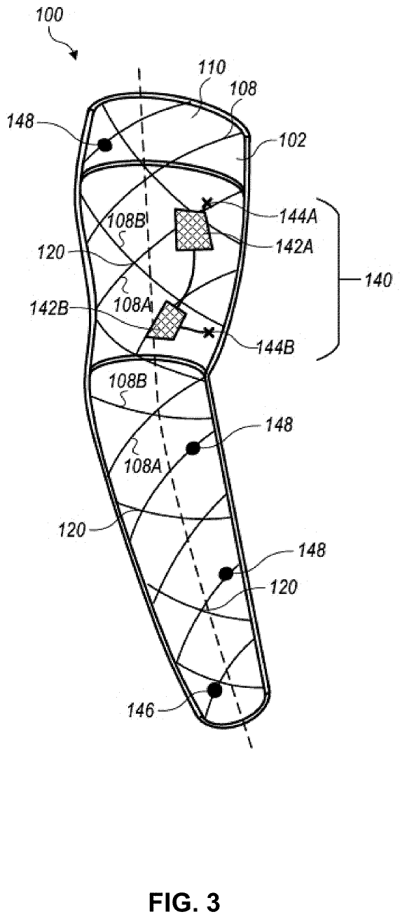

FIG. 3 depicts a lateral cut-away representation of an embodiment of sleeve 100. FIG. 3 depicts a view of an interior of sleeve 100 (e.g., the portion of the sleeve that contacts the skin of the limb of the wearer of the sleeve). In certain embodiments, at least one layer of fabric 102 includes an interspersed (e.g., interlaced or interwoven) pattern of different materials. For example, as shown in FIG. 3, fabric 102 may include a geometric (e.g. mesh) pattern of material 108 and material 110. In certain embodiments, material 108 is a conductive material (e.g., a conductive elastic material) and material 110 is a non-conductive material (e.g., a nonconductive elastic material). Material 108 may be, for example, conductive polymer (such as, but not limited to, an elastic conductive polymer), conductive fiber, and/or conductive fabric wiring, any other suitable material, or a combination thereof. In some embodiments, material 108 may exhibit characteristics similar to a metal rubber. Conductive fibers may be in the form of one or more yarns woven or knit with other fibers. In some embodiments, material 108 may be coated with an insulative material (e.g., an insulative polymer) to provide more efficient conductive properties (e.g., less current or signal leakage).

In some embodiments, material 108 is interspersed into material 110 (e.g., material 108 is laced into or woven into material 110). For example, material 108 may be fibers or wiring engrained or partially engrained within seams of sleeve 100 (e.g., within seams of material 110). In some embodiments, material 108 and material 110 are formed as “panels” of fabric 102. For example, material 108 (e.g., the conductive material) may be used as borders for panels of material 110 (e.g., the non-conductive material). The panels may then be interspersed (e.g., interlaced or interwoven) together to form the geometric pattern shown in FIG. 3.

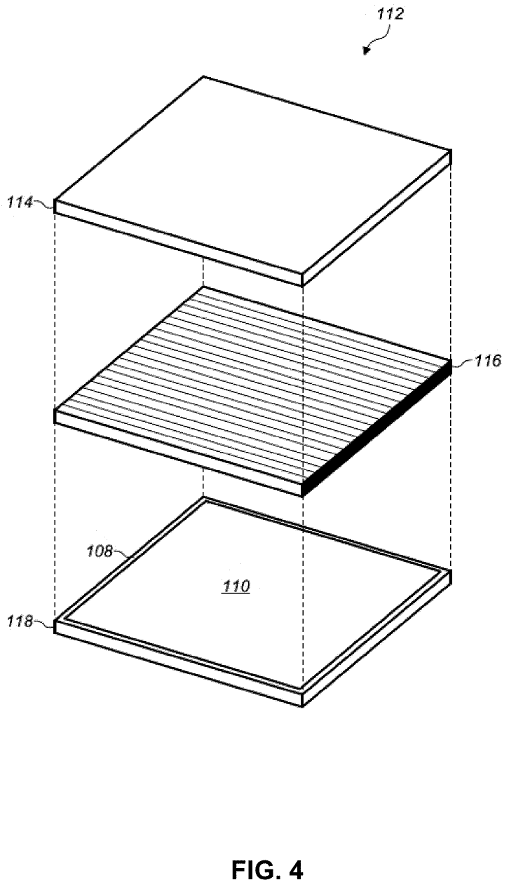

In some embodiments, the panels are made of multi-layers of fabric. FIG. 4 depicts an exploded side view of an embodiment of multi-layer fabric panel 112. Panel 112 may include top layer 114, midsection 116, and bottom layer 118. It is to be understood that while multi-layer panel 112 is depicted in FIG. 4, the multiple layers of fabric may also be used in other embodiments of fabric 102 that are not formed from panels.

In certain embodiments, bottom layer 118 includes material 108 (e.g., the conductive material) as borders for material 110 (e.g., the non-conductive material). Material 108 may be, for example, conductive polymer, conductive flexible fibers, or another conductive flexible (elastic) material. In some embodiments, material 108 includes a rubberized conducive material, such as, but not limited to a metal rubber. Metal rubber may provide an ideal set of properties including elasticity and conductivity. When an individual is wearing sleeve 100, bottom layer 118 may be adjacent the individual's skin Material 108 in bottom layer 118 may receive a natural current from the individual's skin that may be transmitted throughout the bottom layer. In certain embodiments, as described herein, this natural current may be measured by one or more sensor units, which may output data that is assessed to show the shape of the limb.

Midsection 116 may be an insulative fabric such as, but not limited to, a woven textile including insulative fibers. In some embodiments, midsection 116 may include crosslinked material. It should be noted that the insulative fibers of the woven textile may be adjacent bottom layer 118 so that the bottom layer may carry a charge from one point to another without midsection 116 interfering with the current passed through the bottom layer. Top layer 114 may include an elastic fabric such as, but not limited to spandex and Lycra. In certain embodiments, midsection 116 is adhered to the top and bottom layers 114, 118 via an adhesive polymer. In some embodiments, the woven textile of midsection 116 is woven to at least one of the top and bottom layers 114, 118.

In certain embodiments, as shown in FIG. 3, sensor units 120 are located at intersections of material 108 in sleeve 100. The intersections may be vertices in the geometric pattern (e.g., contact points between different paths of material 108 and/or overlap points between different paths of material 108). The geometric pattern of material 108 (e.g., the conductive material) may provide portions 108A that are oriented in a first direction and portions 108B that are oriented in a second direction. Portions 108A may be oriented substantially perpendicular to portions 108B or at other angles (e.g., 45° or 60°) to portions 108B. As material 108 (including portions 108A, 108B) is integrated in sleeve 100 and positioned over muscular areas of the wearer's limb, the material stretches and contracts as the muscles of the limb expand and contract (e.g., material 108 and fabric 102 expands and contracts along with motion of the muscles).

In certain embodiments, sensor units 120 detect the changes in the resistance of material 108 that results from the expansion and contraction of the muscles in the limb. Material 108 may have a defined length and width (e.g., area) between two sensor units 120. With the defined (known) area of material 108 between two sensor units 120, the sensor units may assess changes in shape and/or motion of the limb (e.g., expansion and/or contraction of muscles in the limb) based on assessed changes in the resistance of the material.

In some embodiments, sensor units 120 assess resistance (and changes in resistance) of material 108 between sensor units by assessing (e.g., measuring) electrical currents passing through the sensor units. For example, sensor units 120 may assess currents passing through the sensor units along portions of material 108 connected to the sensor units. Resistance may then be determined from the assessed (measured current).

In some embodiments, transmission time between sensor units 120 may be used to assess resistance changes in material 108 between sensor units. For example, while wearing sleeve 100, changes in shape and/or motion of muscles in the user's limb may cause material 108 to elongate and conductive fibers in the material to become uniformly thinner. As the conductive fibers become thinner, the resistance along the conductive fibers increases. Because of the increased resistance, the transmission time of the electrical signal across material 108 increases. These transmission times may be recorded in sensor units 120 placed within sleeve 100. The recordation of transmission times may be included in information/data packets sent to processor 130 as described herein.

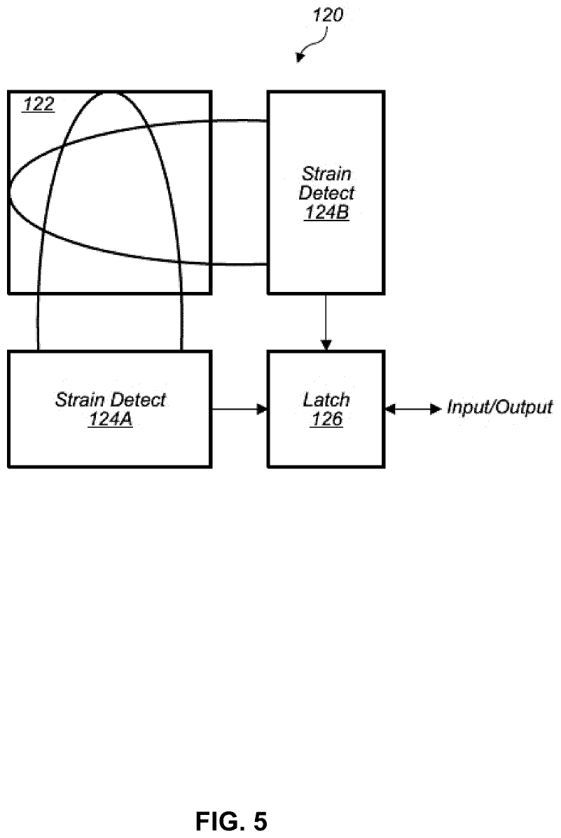

In some embodiments, sensor units 120 assess change in resistance of material 108 using strain detection. FIG. 5 depicts a representation of an embodiment of sensor unit 120. Sensor unit 120 may include conductive material panel 122, multiple instances of strain detection unit 124 (e.g., strain detection units 124A, 124B), and latch 126. Panel 122 may include at least 2 strips of material 108 or another conductive material with the strands in substantially perpendicular directions (e.g., one substantially in the horizontal direction and one substantially in the vertical direction as depicted). The material in panel 122 stretches or relaxes with expansion and contraction associated with motion of muscles in the limb.

As the material in panel 122 includes conductive material (e.g., conductive polymer or conductive fiber), the resistance of the conductive material changes. When the area of the conductive material decreases, its resistance increases. Deflection (e.g., expansion and contraction) of the conductive material results in a decrease in the cross-sectional area and a corresponding change in the resistance of the conductive material.

Strain detection units 124A and 124B may detect the changes in the resistance of the conductive material in panel 122 that results from the stretching or relaxing of the material that accompanies expansion and contraction of muscles in the limb. Latch 126 may capture the results of the detection performed by strain detection units 124A, 124B. Data captured by latch 126 may be provided to, for example, processor 130, as described herein.

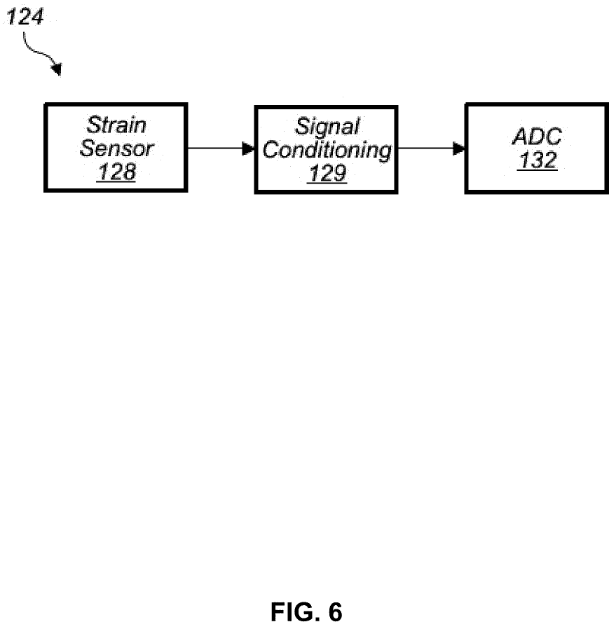

FIG. 6 depicts a representation of an embodiment of strain detection unit 124. Strain detection unit 124 may include strain sensor unit 128, signal conditioning unit 129, and analog-to-digital converter (“ADC”) 132. During operation, strain sensor unit 128 may detect the changes in resistance resulting from the deflection of the conductive material (e.g., material 108) strands due to expansion and/or contraction of muscles in the limb. The results from strain sensor unit 128 may be provided to signal conditioning unit 129, where the resulting signal or signals are, for example, amplified and any DC offset may be removed. The conditioned signal may be provided to ADC 132 where the signal is converted into a digital output. ADC 132 may be a simple 1-bit ADC, a more complex 24-bit ADC, or something in between, depending upon the application and the needs of the system. The digital output may be provided to processor 130 as output from sensor unit 120, as described herein.

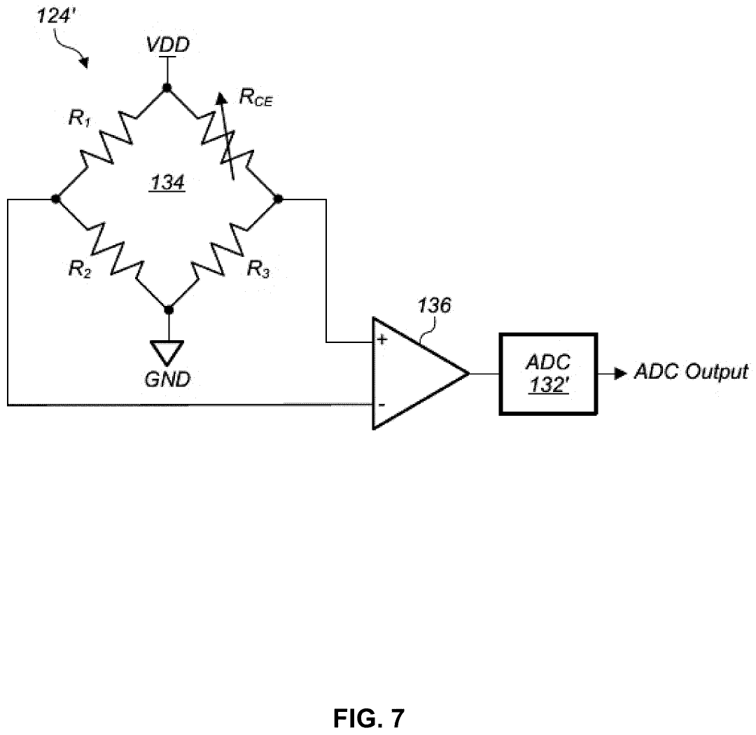

FIG. 7 depicts a representation of another embodiment of strain detection unit 124′. Strain detection unit 124′ may include Wheatstone bridge 134, amplifier 136, and ADC 132′. Wheatstone bridge 134, as is known in the art, may be often used to accurately measure small changes in resistance of a strained medium, converting the changes in resistance into a voltage that can be amplified by amplifier 136 and converted to a digital output by ADC 132′. In certain embodiments, Wheatstone bridge 134 includes four resistors R1, R2, R3, and RCE, where RCE is the resistance of the conductive material (e.g., material 108). When all four resistors in Wheatstone bridge 134 are equal, the bridge may be perfectly balanced and the output voltage is equal to zero. But when any one or more of the resistors change value by only a fractional amount, the bridge produces a measurable voltage.

Thus, when the resistance of the material, illustrated here as RCE, changes, the output voltage provided to amplifier 136 reflects that change as a change in voltage which is then conditioned and amplified by amplifier 136. The amplified signal is then converted to a digital output by ADC 132′. As before, ADC 132′ may be a simple 1-bit ADC, a more complex 24-bit ADC, or something in between, depending upon the application and the needs of the system.

In certain embodiments, sensor units 120 generate one or more data packets with the assessed resistance data associated with material 108 in sleeve 100. The assessed resistance data may include, for example, resistance measurements themselves as assessed by sensor units 120. In some embodiments, the resistance data in the data packets may include data that can be used to determine the resistance data of material 108. For example, the resistance data may include current measurements, transmission time measurements, or digital voltage outputs, as described above. In certain embodiments, the data packets may include time stamps for the measurements and/or identification information for sensor units 120 (e.g., a unique ID associated with the sensor unit sending the data packet). Sensor units 120 may send the data packet(s) to either a processor integrated in sleeve 100 (e.g., processor 130, as shown in FIG. 2) or an external processor. In some embodiments, sensor units 120 send the data packet to the processor integrated in sleeve 100 using material 108 (e.g., via a conductive wired path in the sleeve). Sensor units 120 may also be capable of transmitting the data packet wirelessly (e.g., using Bluetooth or another wireless data transmission technique).

In certain embodiments, sleeve 100 includes processor 130, as described herein and shown in FIG. 2. Processor 130 may be integrated in sleeve 100. For example, processor 130 may be integrated in a layer of fabric 102 or between layers in the fabric. Processor 130 may provide processing of information acquired through various sensors/components on sleeve 100 (e.g., inertial measurement units 106 and/or sensor units 120). Processor 130 may process the acquired information (e.g., raw data from sensors/components) to generate new information. In some embodiments, processor 130 is coupled to memory 138. Memory 138 may be used for storing the information and processing or transmitting the information at a later time. Memory 138 may also be used for storing instructions or other process protocols as described herein.

In certain embodiments, wireless transmitter 140 is coupled to processor 130 and memory 138. Wireless transmitter 140 may be capable of wireless transmission/receiving using a wireless transmission protocol. Wireless transmission protocols utilized by wireless transmitter 140 may include, but not be limited to, cellular transmission, satellite transmission, Bluetooth (including different variations of Bluetooth transmission protocols), ANT+, Zigbee, Wi-Fi, LiFi, and SATCOM. Transmitted information may include either processed information and/or raw data information. In some embodiments, wireless transmitter 140 provides burst transmission of information (e.g., transmits bundles of information at a specified time).

As shown in FIG. 2, in certain embodiments, wireless transmitter 140 is separate from and attached to processor 130. Wireless transmitter 140 may be integrated in sleeve 100 (e.g., integrated in or between layers of fabric 102). In some embodiments, wireless transmitter 140 is part of processor 130. In some embodiments, antennas for wireless transmission/reception are integrated in sleeve 100 and coupled to wireless transmitter 140. For example, flexible, flat antennas may be integrated into fabric 102 of sleeve 100. Integration of the antennas may include sewing or embedding the antennas into fabric 102. The antennas may include small circuit boards using lightweight materials that provide fast data transfer rates.

In some embodiments, wireless transmitter 140 provides for the addition of third-party hardware for extended biofeedback response capabilities. Some possible hardware concepts include, but are not limited to, glasses to track movement and pupil dilation, wristbands to monitor skin conductivity and temperature, ambient temperature sensors, and DTR (deep tendon reflex) monitoring. In some embodiments, wireless transmitter 140 includes small transmitters and receivers capable of moving large quantities of data and smaller package sizes over greater distances. Wireless transmitter 140 may utilize protocols to include a wide variety of third-party hardware. In some embodiments, submersible technology may be incorporated in wireless transmitter 140.

As described above, processor 130 may receive data (e.g., information) from inertial measurement units 106 and/or sensor units 120. In certain embodiments, processor 130 receives the data from inertial measurement units 106 and/or sensor units 120 via wireless transmission (e.g., through wireless transmitter 140). In some embodiments, processor 130 receives the data via wired transmission. For example, inertial measurement units 106, sensor units 120, and processor 130 may be coupled to material 108 (e.g., the conductive material). Material 108 (e.g., wiring in sleeve 100) may then be used to transmit/receive data between inertial measurement units 106, sensor units 120, and processor 130. In some embodiments, separate wiring in sleeve 100 may be used to transmit receive data between inertial measurement units 106, sensor units 120, and processor 130.

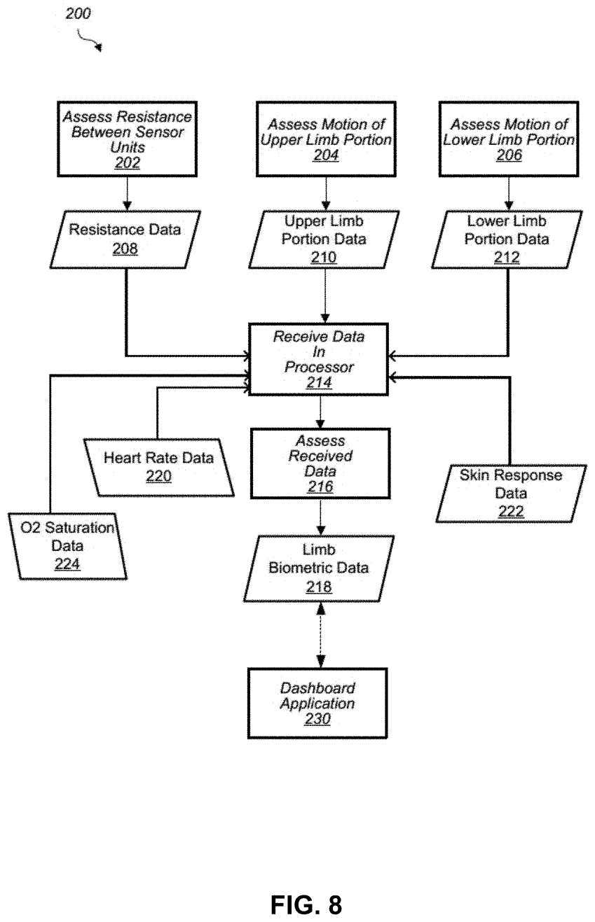

Data received by processor 130 may be used to determine biometric properties of the user's limb associated with sleeve 100. FIG. 8 depicts a flowchart of an embodiment of method 200 of determining biometric properties of a user's limb using sleeve 100. In certain embodiments, method 200 includes assessing resistance between sensor units in 202, assessing motion of the upper portion of the limb in 204, and assessing motion of the lower portion of the limb in 206.

As described above, assessing resistance between sensor units in 202 may include assessing the resistance (or changes in resistance) of material 108 between sensor units 120, shown in FIG. 3. Further as described above, resistance data 208 from the assessing in 202, shown in FIG. 8, may include actual resistance data or data that can be used to determine resistance (e.g., current measurements used to determine resistance). In embodiments where resistance data is data used to determine resistance, processor 130 may determine the resistance data (e.g., as part of “assess received data 216” described below).

As described above, assessing motion of the upper portion of the limb in 204 may include measuring motion of the upper portion of the limb with inertial measurement unit 106A, which is above positioning component 104, as shown in FIGS. 1 and 2. Output from the assessing in 204 may be provided as upper limb portion data 210. Assessing motion of the lower portion of the limb in 206 may include measuring motion of the lower portion of the limb with inertial measurement unit 106B, which is below positioning component 104. Output from the assessing in 206 may be provided as lower limb portion data 212. Upper limb portion data 210 and lower limb portion data 212 may include, as described herein, motion data measured with multiple degrees-of-freedom (e.g., nine degrees-of-freedom).

As shown in FIG. 8, resistance data 208, upper limb portion data 210, lower limb portion data 212 may be provided to process 130 in “receive data in processor 214”. The data may be provided from each component (e.g., inertial measurement units 106 and/or sensor units 120) to processor 130 using wireless or wired techniques described herein. After the data is received in processor 130, the processor may assess the received data in 216. In certain embodiments, processor 130 assesses the received data and generates limb biometric data 218. Limb biometric data 218 may include, for example, shape, position, and/or movement information about the limb wearing sleeve 100. In certain embodiments, limb biometric data 218 includes full motion of the limb including motion of the shoulder joint (e.g., glenohumeral joint), the elbow joint, the wrist joint, and muscles in the limb. Full motion data may include: flexion, extension, adduction, abduction, and rotation of the glenohumeral joint; flexion and extension of the elbow joint; and pronation and supination of the wrist joint. Full motion of the muscles may include changes in shape of the muscles (e.g., circumferential changes in the muscles) in the upper arm and the humerus. In some embodiments, limb biometric data 218 includes other measurement data described herein (e.g., heart rate data 220, skin response data 222, 02 saturation data 224).

In certain embodiments, limb biometric data 218 includes multi-dimensional images of the shape, position, and/or movement of the limb in sleeve 100. For example, the combination of measurements from portion 108A and portion 108B of material 108, shown in FIG. 3, may be used to generate an image of muscle motion (e.g., changes in shape, position, and movement of the muscles caused by muscle expansion/contraction) of the limb of the wearer of sleeve 100. As described above, portion 108A may be in a first direction and portion 108B may be in a second direction. Thus, portion 108A may be used to assess muscle motion in the first direction while portion 108B is used to assess muscle motion in the second direction. Assessing muscle motion in two different directions may be used to generate a multi-dimensional image of the muscle motion in the limb (e.g., changes in shape, position, and movement of the muscles caused by expansion and/or contraction of muscles). The muscle motion assessed using portions 108A, 108B of material 108 may be combined with other measurements (e.g., inertial measurement units 106) to provide a three-dimensional image of motion of the limb. For example, the three-dimensional image may include an image of expansion and/or contraction of muscles in the limb assessed by material 108 along with movement of the limb and portions of the limb itself assessed by inertial measurement units 106.

As described above, sleeve 100 may be capable of measuring motion of the limb with nine degrees-of-freedom both above and below the joint location of the sleeve along with relative motion between portions of the limb above and below the joint location using inertial measurement units 106A and 106B. Data from inertial measurement units 106A and 106B may be combined with data from sensor units 120 to provide a three-dimensional image of the motion of the limb including shape, position, and movement of the limb in space. In some embodiments, the three-dimensional motion image may be combined with other measurements possible with sleeve 100 described herein (e.g., heart rate data 220, skin response data 222, 02 saturation data 224) to provide a biometric assessment of the wearer of sleeve 100 in limb biometric data, as shown in FIG. 8. For example, the biometric assessment of the wearer may include assessment motion of the wearer based on the assessed shape, position, and movement of the limb of the wearer in association with the other measured properties. Thus, sleeve 100 may provide an assessment of substantially every aspect of motion of the limb including, but not limited to, gait mechanics and movement of the limb. Gait mechanics may include upper-body gait mechanics such as symmetry, smoothness, variability, and stability as well as limb (arm) swing amplitude and axial rotation. Limb movement may include motion at the shoulder, elbow, and wrist joints.

In certain embodiments, as shown in FIG. 3, sleeve 100 includes heart rate monitor 140. Heart rate monitor 140 may include first lead 142A and second lead 142B. First lead 142A and second lead 142B may be positioned on the inside of sleeve 100 (e.g., inside surface of fabric 102) such that the leads are in contact with the skin of the wearer when the sleeve is worn on the wearer's limb. In certain embodiments, first lead 142A and second lead 142B are coupled to processor 130 using wired connections. For example, first lead 142A may be coupled to a wire that passes through first passthrough 144A in fabric 102 and second lead 142A may be coupled to a wire that passes through second passthrough 144B in the fabric. The wires going through first passthrough 144A and second passthrough 144B may then couple to processor 130. In some embodiments, wires for first lead 142A and second lead 142B may be made from portions of material 108 (e.g., the conductive material) in fabric 102.

Heart rate monitor 140 may be, for example, a standard heart rate monitor that circumnavigates either a portion of the user's limb or torso. Heart rate monitor 140 may function utilizing decoding algorithms and EKG equivalent monitoring techniques. In some embodiments, heart rate monitor 140 may integrate a three lead EKG equivalent monitor (e.g., a lead measured between three electrode sites (+, −, and ground). Heart rate monitor 140 may, however, integrate any number of leads. Heart rate monitor 140 may track pulse rate (and/or other heart related information) in near real-time. Heart rate monitor 140 may stream pulse rate or other data to processor 130 (e.g., via wires as described above) as heart rate data 220, shown in FIG. 8. Processor 130 may run the data through multiple algorithms developed and offered as a package. The algorithms offered may provide, but not be limited to, the following outputs: BPM (beats per minute), HRR (heart rate reserve), stress response, and HRV (heart rate variability).

In certain embodiments, as shown in FIG. 3, sleeve 100 includes oxygen sensor 146. Oxygen sensor 146 may be, for example, a pulse oximeter. Oxygen sensor 146 may be used to assess SpO2 (blood oxygen saturation) levels in the limb of the wearer. In some embodiments, oxygen sensor 146 is included with heart rate monitor 140 in sleeve 100. Oxygen sensor 146 may be coupled to processor 130 using material 108 (e.g., the conductive material). Measurements of SpO2 from oxygen sensor 146 may be provided to processor 130 as O2 saturation data 224, shown in FIG. 8. O2 saturation data 224 may be combined with other data by processor 130 to provide an assessed condition of the wearer as described herein. In some embodiments, oxygen sensor 146 is removably attached to sleeve 100. For example, oxygen sensor 146 may be attached and removed from sleeve 100 as desired for use (e.g., based on the needs of the wearer of the sleeve).

In certain embodiments, as shown in FIG. 3, sleeve 100 includes one or more skin response sensors 148. Skin response sensors 148 may be, for example, galvanic skin response sensors. Skin response sensors 148 may be used to assess skin conductivity, skin temperature, or other skin properties. For example, skin response sensors 148 may be used to assess changes in electrical activity resulting from changes in sweat gland activity in the skin Skin response sensors 148 may be coupled to processor 130 using material 108 (e.g., the conductive material). Measurements of skin response from skin response sensors 148 may be provided to processor 130 as skin response data 222, shown in FIG. 8. Skin response data 222 may be combined with other data by processor 130 to provide an assessed condition of the wearer as described herein. In some embodiments, skin response sensors 148 are removably attached to sleeve 100. For example, skin response sensors 148 may be attached and removed from sleeve 100 as desired for use.

In some embodiments, sleeve 100 includes a GPS component. The GPS component may be included as part of processor 130 or another component in sleeve 100. The GPS component may include, for example, a GPS monitor and/or a WWAN monitor. The GPS monitor may be stacked or swapped with the WWAN monitor for indoor movement tracking in a 3D space. The GPS component may be utilized to track the physical location of the limb (and the wearer's body) in a real-time environment. For certain purposes (e.g., military purposes), the GPS component may be capable of utilizing WWAN to track a wearer through an interior environment. WWAN integration may afford observers utilizing a dashboard application (as described herein) to track the sleeve's wearer in near real time on a map overlay. In some embodiments, the GPS component includes one or more small units that provide good satellite tracking, fast locking, and good transmission through dense cover.

In certain embodiments, as shown in FIG. 8, processor 130 sends limb biometric data 218 to dashboard application 230. Dashboard application 230 may be an application associated with sleeve 100. For example, dashboard application 230 may be an application or module that is associated with sleeve 100 and operating on device 300, as shown in FIG. 2. Device 300 may be, for example, a mobile device or other electronic device. In certain embodiments, device 300 communicates with processor 130 via wireless transmitter 140 using communication protocols described herein. Processor 130 may communicate with device 300 to send/receive data between dashboard application 230 and sleeve 100.

Dashboard application 230 may be associated with sleeve 100 to provide simultaneous review of all biometric information assessed by the sleeve as well as complementary information generated by the processing of acquired data (e.g., algorithmic manipulation of acquired data). Dashboard application 230 may allow for the management, utilization, and near real-time review of gathered data regardless of the physical location of device 300 relevant to sleeve 100. In some embodiments, dashboard application 230 is a native iOS or Android application as well as a web platform. In some embodiments, dashboard application 230 may provide additional processing of data received from sleeve 100.

Dashboard application 230 may be capable of accessing a remote server associated with sleeve (e.g., through a secure Internet connection). Dashboard application 230 may provide capability for the passing of information and system management tools between sleeve 100 and the remote server. This setup may allow for wireless firmware updates and remote diagnostic capabilities of sleeve 100. Live “over-the-wire” firmware updates may occur as enhancements are made and process 130 may be updated as improvements occur. Initially, dashboard application 230 may allow for the measurement and viewing of all biometric processes being monitored and GPS location.

In some embodiments, communication between processor 130 and dashboard application 230 is substantially continuous (e.g., limb biometric data 218 is continuously transmitted to the dashboard application by the processor). In some embodiments, processor 130 may provide burst transmission of data to dashboard application 230. For example, processor 130 may store data in memory 138 in the event of communication loss between the wireless transmitter 140 and device 100. Upon reestablishment of the communication link, burst transmission of the data may be provided to update dashboard application 230 with data as quickly as possible.

In certain embodiments, dashboard application 230 may display data received from processor 130 and/or information generated from the data received from the processor on a display of device 300. Dashboard application 230 may display data such as, but not limited to, limb biometric data 218, information generated from the limb biometric data, and other data obtainable from sleeve 100 (e.g., GPS data). Dashboard application 230 may also store data received on device 300 so that data history for sleeve 100 (and the wearer of the sleeve) can be accessed at later times. In some embodiments, dashboard application 230 may transmit data for storage on a remote server (e.g., a cloud-based server).

As described herein, sleeve 100 may provide an effective system for tracking movement of a limb along with an array of biometric properties associated with the limb. Sleeve 100 may, for example, provide tracking of gait mechanics of the limb, limb movement, heart rate, and autonomic tone. Tracking of these properties may be substantially simultaneous using sleeve 100. Tracking of these properties may also be provided in real-time as the limb is being used.

Sleeve 100, as described herein, may be used in a range of applications including medical applications. Medical applications may include, but not be limited to, fall predication and early diagnosis of Parkinson's disease. Sleeve 100 may also provide capability for three-dimensional computer modeling of the limb, sports performance tracking, and/or biofeedback training.

### NON-LIMITING EXAMPLES

The following non-limiting examples provide different embodiments of use of sleeve 100.

### First Example Embodiment

Sleeve 100 may be used to detect signs associated with neuromuscular diseases such as, but not limited to, amyotrophic lateral sclerosis, multiple sclerosis, myasthenia gravis, neuromuscular junction disease, spinal muscular atrophy, and Parkinson' disease. In an example, a 60-year old male may be doing an annual physical with a physician. The male subject may put on sleeve 100 during his exam and wear the sleeve while walking around the room. Data obtained by sleeve 100 may show a decrease in arm swing and axial rotation amplitude, with less smoothness than the previous year. The trend shown by sleeve 100 may indicate a potential path towards Parkinson's disease in later years.

By identifying the indicators from sleeve 100 early on, the physician may propose that further testing is necessary as there may be a high probability of Parkinson's disease in the future. The physician may also start early treatment to slow onset and progression of Parkinson's disease. Typically, the earliest features of Parkinson' disease develop slowly and begin to manifest years before a formal diagnosis is possible. Early identification and treatment may be a critical factor in managing the disease, easing its progression, and providing the patient with a more satisfying quality of life.

Reduced arm swing during gait has long been a classical marker of Parkinson's disease. Recent research has focused on using gait analysis to identify variations in arm swing and axial rotation that preclude the onset of Parkinson's disease while the disease is still in the prodromal stage, prior to when a formal diagnosis can be made. For example, the disease susceptibility may be assessed in otherwise healthy people via gait analysis using sleeve 100. This may allow for more rapid identification of high-risk individuals. Earlier identification of Parkinson's disease susceptibility may allow for earlier intervention, which can mitigate and slow progression of the disease.

During gait analysis, the two primary indicators of prodromal motor features (the earliest signs of future Parkinson's disease) are decrease arm swing amplitude and axial rotation. Arm swing amplitude is the total distance that each arm reaches forward and back while the subject is waling. Axial rotation is the amount of rotation occurring at the thoracic spine combined with protraction and retraction (reaching forward and pulling back) of the shoulder blade across the ribcage. Side to side asymmetry of these movements may be another indicator of prodromal status.

Sleeve 100 may enable detailed measurement of both arm swing amplitude and axial rotation (as well as other limb-based factors). For example, inertial measurement units 106 may assess transverse, frontal, and sagittal plane arm swing amplitude as well as elbow flexion/extension and wrist rotation during gait. At the same time, sensor units 120 may assess axial rotation by tracking the reciprocal amplitude of rotation at the shoulder of each arm.

Sleeve 100 may provide this limb biometric data, including heart rate variability, for analysis by the physician or another research. The analysis may be used to observe variations in gait mechanics that preclude Parkinson's disease as well as other potential indicators such as aberrant heart rate variability and changes in SPO2 saturation. Sleeve 100 may allow a more complete picture of the inner and outer workings of the subject's body and provides a better picture into the body's physiological interactions. Thus, sleeve 100 may provide enhanced ability for early detection of Parkinson's disease and allow for earlier treatment to provide a more positive impact on the lifespan and quality of life of the subject.

### Second Example Embodiment

A software designer may be attempting to use digital 3D (three-dimensional) modeling to create images of a person's body in motion. Sleeve 100 may be used to track detailed three-dimensional motion of motion of the person's limbs in an easy and accurate manner. For example, a model may wear one or more sleeves 100 and go through motions needed by the software designer. The data stream (e.g., stream of biometric data) from sleeves 100 may show shoulder, elbow, and wrist motion of the arms while also displaying changes in the shapes of muscles in the arm as the arms move.

### Third Example Embodiment

A rugby player may be training in a weight room. The player may be concerned with shorter-term physical performance along with building resilience to reduce the risk of injury. Years of conventional gym training may have created lots of strength and tension in the sagittal plane extension—i.e., the player is good at squatting, deadlifting, and pressing. This tension bias into a single plane of motion may, however, have created some pattern rigidity. For example, the player may have tight extensors, such as muscles in the lower back, and the player's ability to dynamically change direction and move laterally and rotationally on the field may have decreased. Thus, while the player may be better at picking up a barbell, the player may be falling behind competitively in many aspects of the sport due to deterioration of movement availability on the field. The player may be more achy, prone to small injuries, and not recovering as well because the player cannot turn off the tension that the body has been training towards for years.

To overcome these issues, the player may use sleeve 100 to track training movements and adjust the player's training to incorporate more lateral and transverse plane movement. Such movement may have a particular focus on alternating reciprocal motion at the arms and thorax during movements like lunges, step-ups, and single-arm presses or rows. Using sleeve 100 in training at the gym to focus on these movements, the player may now incorporate natural thoracic rotation and scapulae/arm reach found in running and jumping. Sleeve 100 may be used to monitor and assess these movements. Sleeve 100 may track how well the player is moving rotationally during strength movements and, potentially, provide haptic feedback to correct the movement if the player falls into old training patterns. Additionally, sleeve 100 may be tracking other biometric properties such as heart rate to help monitor exertion and recovery during the workouts.

### Fourth Example Embodiment

A 70-year old female may potentially be having issues with balance and is worried about falling. The female may utilize sleeves 100 to monitor her gait and warn of impending falls. Sleeves 100 may, for example, monitor for sudden changes in gait mechanics including stability, variability, smoothness, and symmetry as well as arm swing amplitude and axial rotation. Sleeves 100 may simultaneously monitor autonomic tone via changes in heart rate variability that could indicate a shift towards a threatened or highly stressed state. Each of these properties may be positively correlated with falls in the elderly and be used to predict a fall.

The female may wear sleeves 100 during normal daily life patterns. She may not normally notice sleeves 100 until she is alerted that a shift in her movement indicates a probability of a fall. The alert triggered by sleeves 100 may focus her attention to her movement and make her realize that she is feeling more unstable than normal. She may then immediately seek to rest (e.g., sit down) for a moment to regain her normal stability. Heart rate and heart rate viability tracking in sleeves 100 may provide her an indication (e.g., after a few minutes of rest) that she has stabilized and that she may safely continue with her activity.

### Fifth Example Embodiment

A 27-year old climber had his left biceps muscles removed following a traumatic fall that was untreated for several hours. His humerus, though fractured, was intact. A surgical team performed a latissimus dorsi transposition for a biceps reconstruction. There were no complications during or after the surgery. The goal of medical treatment and rehabilitation after the surgery is to restore as much function as possible to his left arm. Major upper arm amputations require functional restoration of elbow flexion. Sleeve 100 may be used during the rehabilitation of the biceps to contrast and compare normal biceps biometric data with the data for the reconstructed biceps. This data may be used to guide functional restoration of the reconstructed biceps.

### Sixth Example Embodiment

A 42-year old former police officer may be having episodes of anxiety and hypervigilance. A psychiatrist may recommend that the officer use sleeve 100 to track the frequency of the episodes and to monitor changes in vital signs during the episodes. The officer and the psychiatrist may be working on controlling the episodes at the beginning. Sleeve 100 may provide data to dashboard application 230 that includes a biofeedback process that the patient can integrate into previously learned interventions such as breathing or meditation drills upon early onset of the anxiety. Sleeve 100 may be used to assess efficacy of the interventions and allow for assessment or change to the psychiatric care of the officer.

### Seventh Example Embodiment

An account executive may have poorly controlled blood pressure and be frequently travelling for work. The executive may not have time to go to walk-in blood pressure clinics or find other means for monitoring blood pressure. The executive may also forget to take his/her blood pressure measurement regularly. Sleeve 100 may be used to monitor blood pressure periodically and track results on his/her mobile device. The results tracking may be used by the executive to self-monitor and/or the information may be shared with his/her physician for additional tracking.

### Eighth Example Embodiment

A 15-year old Type-1 diabetic may use sleeve 100 to non-invasively monitor his/her blood glucose level. For example, a blood glucose monitor may be integrated into sleeve 100. When his/her glucose level is assessed to be outside of an acceptable range, sleeve 100 may provide a notification (e.g., through dashboard application 230) that insulin should be administered. In some cases, sleeve 100 may be associated with an automatic insulin injector assembly to provide an emergency insulin dose if the glucose level rises to an urgent level. An example of an automatic insulin injector assembly is disclosed in U.S. patent application Ser. No. 16/049,093 to Bogdanovich et al., which is incorporated by reference as if fully set forth herein.

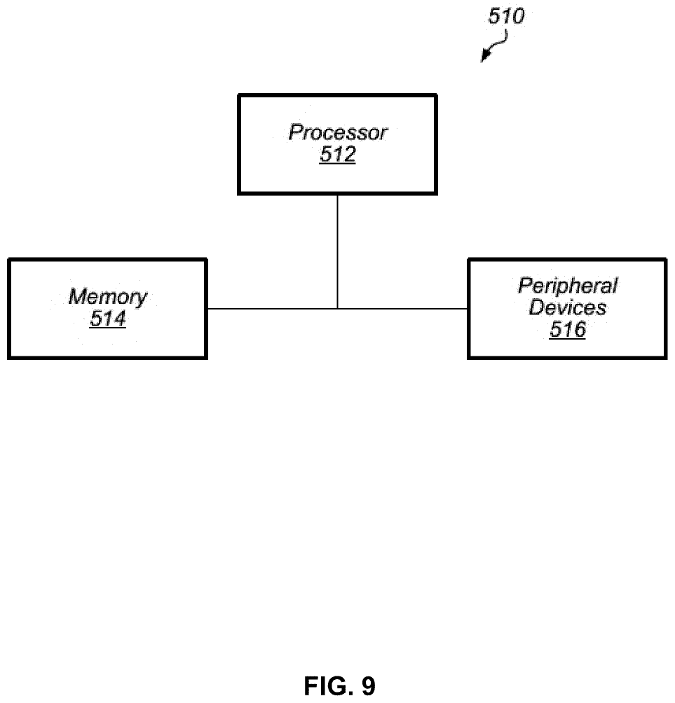

FIG. 9 depicts a block diagram of one embodiment of exemplary computer system 510. Exemplary computer system 510 may be used to implement one or more embodiments described herein. In some embodiments, computer system 510 is operable by a user to implement one or more embodiments described herein such as communication between processor 114 and a mobile device. In the embodiment of FIG. 9, computer system 510 includes processor 512, memory 514, and various peripheral devices 516. Processor 512 is coupled to memory 514 and peripheral devices 516. Processor 512 is configured to execute instructions, including the instructions for communication between method 200, which may be in software. In various embodiments, processor 512 may implement any desired instruction set (e.g. Intel Architecture-32 (IA-32, also known as x86), IA-32 with 64-bit extensions, x86-64, PowerPC, Sparc, MIPS, ARM, IA-64, etc.). In some embodiments, computer system 510 may include more than one processor. Moreover, processor 512 may include one or more processors or one or more processor cores.

Processor 512 may be coupled to memory 514 and peripheral devices 516 in any desired fashion. For example, in some embodiments, processor 512 may be coupled to memory 514 and/or peripheral devices 516 via various interconnect. Alternatively or in addition, one or more bridge chips may be used to coupled processor 512, memory 514, and peripheral devices 516.

Memory 514 may comprise any type of memory system. For example, memory 514 may comprise DRAM, and more particularly double data rate (DDR) SDRAM, RDRAM, etc. A memory controller may be included to interface to memory 514, and/or processor 512 may include a memory controller. Memory 514 may store the instructions to be executed by processor 512 during use, data to be operated upon by the processor during use, etc.

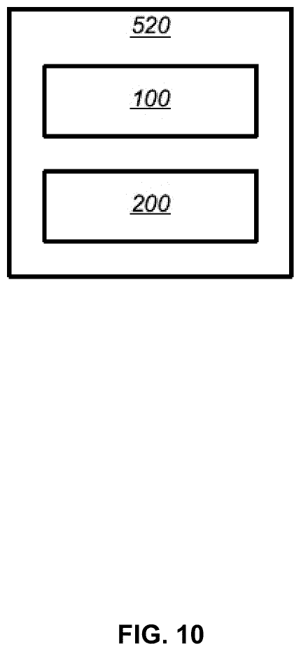

Peripheral devices 516 may represent any sort of hardware devices that may be included in computer system 510 or coupled thereto (e.g., storage devices, optionally including computer accessible storage medium 520, shown in FIG. 10, other input/output (I/O) devices such as video hardware, audio hardware, user interface devices, networking hardware, etc.).

Turning now to FIG. 10, a block diagram of one embodiment of computer accessible storage medium 520 including one or more data structures representative of sleeve 100 included in an integrated circuit design and one or more code sequences representative of method 200. Each code sequence may include one or more instructions, which when executed by a processor in a computer, implement the operations described for the corresponding code sequence. Generally speaking, a computer accessible storage medium may include any storage media accessible by a computer during use to provide instructions and/or data to the computer. For example, a computer accessible storage medium may include non-transitory storage media such as magnetic or optical media, e.g., disk (fixed or removable), tape, CD-ROM, DVD-ROM, CDR, CD-RW, DVD-R, DVD-RW, or Blu-Ray. Storage media may further include volatile or non-volatile memory media such as RAM (e.g. synchronous dynamic RAM (SDRAM), Rambus DRAM (RDRAM), static RAM (SRAM), etc.), ROM, or Flash memory. The storage media may be physically included within the computer to which the storage media provides instructions/data. Alternatively, the storage media may be connected to the computer. For example, the storage media may be connected to the computer over a network or wireless link, such as network attached storage. The storage media may be connected through a peripheral interface such as the Universal Serial Bus (USB). Generally, computer accessible storage medium 500 may store data in a non-transitory manner, where non-transitory in this context may refer to not transmitting the instructions/data on a signal. For example, non-transitory storage may be volatile (and may lose the stored instructions/data in response to a power down) or nonvolatile.

In some embodiments, a wireless device (or wireless station) may be configured to include a processor (or a set of processors) and a memory medium, where the memory medium stores program instructions, where the processor is configured to read and execute the program instructions from the memory medium, where the program instructions are executable to cause the wireless device to implement any of the various method embodiments described herein (or, any combination of the method embodiments described herein, or, any subset of any of the method embodiments described herein, or, any combination of such subsets). The device may be realized in any of various forms.

In embodiments described herein, operating systems utilized by any part of sleeve 100 may include, but not be limited to: iOS operating systems, Windows Phone operating systems, Windows operating systems, Android operating systems, BlackBerry operating systems, Linux systems, and Unison operating systems.

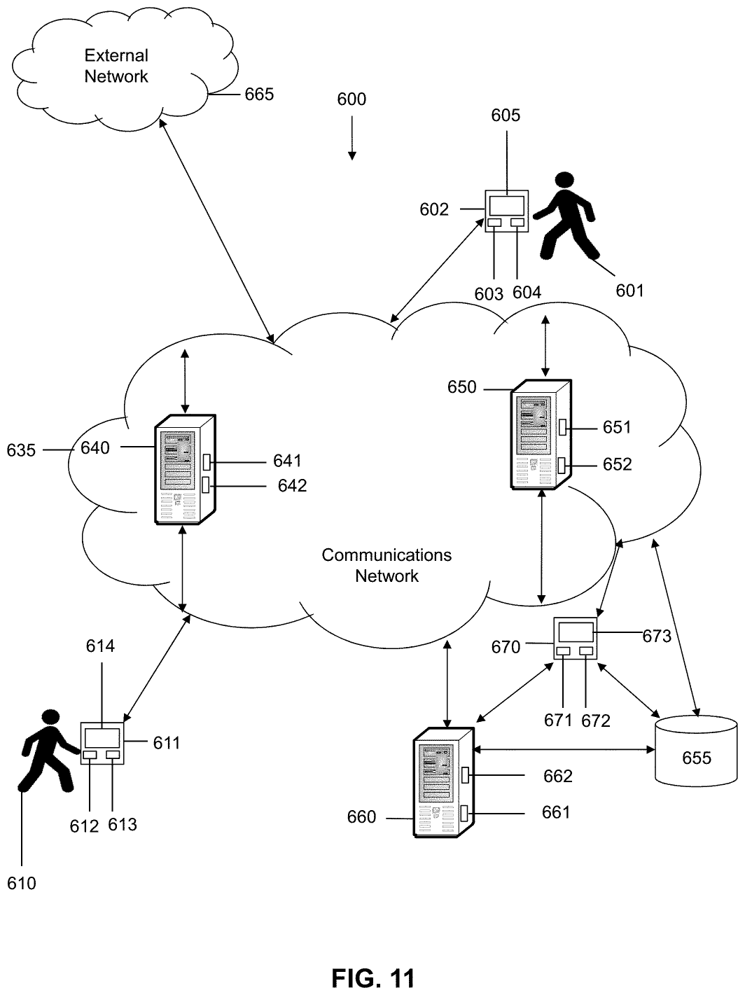

In certain embodiments, the garment sleeve 100, the computing system 510, and/or any of the components of FIGS. 1-10 may be communicatively linked with a system 600, and/or be incorporated into the system 600, as shown in FIG. 11. The system 600 may be configured to perform any of the functionality performed by garment sleeve 100, the computing system 510, and/or any of the componentry of FIGS. 1-10. Additionally, the system 600 may be configured to perform operations and/or functionality offloaded by the garment sleeve 100, the computing system 510, and/or any of the components and processes described in the present disclosure. For example, in certain instances, the computing, storage, and/or other resources of the garment sleeve 100 may be overloaded or may be nearing a threshold level that warrants offloading operations and functionality to the system 600 to assist the garment sleeve 100 in completing various operations and to increase performance of the garment sleeve 100. Notably, any of the components of the garment sleeve 100 may be configured to communicate with any of the components of the system 600, such as via a wired connection, wireless connection, any other type of connection, or a combination thereof. In certain embodiments, the system 600 may form a part of the system encompassing the garment sleeve 100.

The system 600 may be configured to support, but is not limited to supporting, monitoring applications and services, biometric monitoring applications and services, sensor-based applications and services, wearable device applications and services, health monitoring applications and services, communication applications and services, alert applications and services, data and content services, data aggregation applications and services, big data technologies, data synthesis applications and services, data analysis applications and services, computing applications and services, cloud computing services, internet services, satellite services, telephone services, software as a service (SaaS) applications, mobile applications and services, and any other computing applications and services. The system 600 may include a first user 601, who may utilize a first user device 602 to access data, content, and applications, or to perform a variety of other tasks and functions. In certain embodiments, the first user 601 may be a user that is seeking to monitor biometric and/or other information associated with one or more of his body parts and/or his body as a whole. In certain embodiments, the first user 601 may conduct biometric monitoring of himself by utilizing garment sleeve 100.

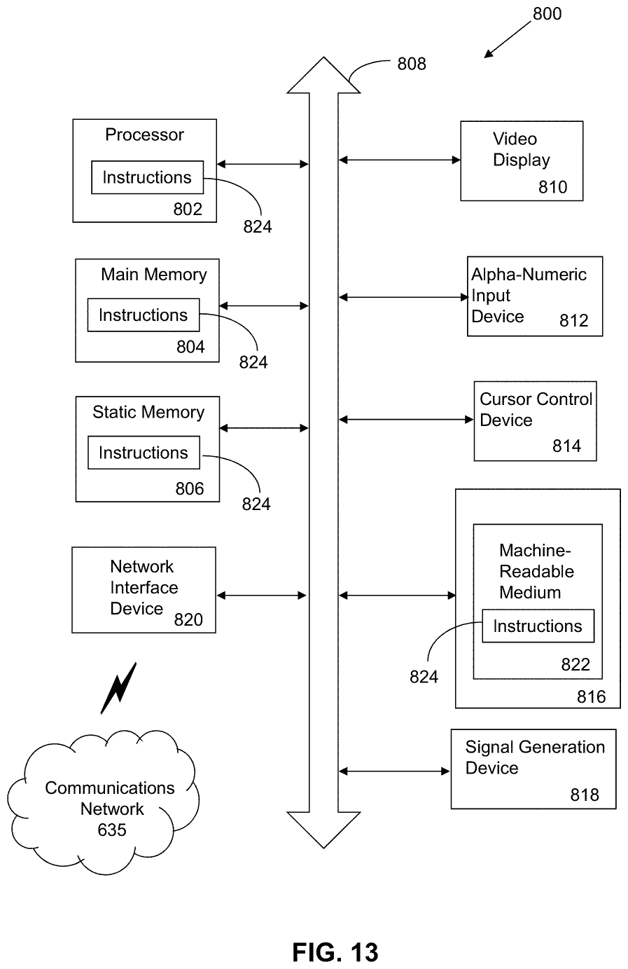

The first user device 602 utilized by the first user 601 may include a memory 603 that includes instructions, and a processor 604 that executes the instructions from the memory 603 to perform the various operations that are performed by the first user device 602. In certain embodiments, the processor 604 may be hardware, software, or a combination thereof. The first user device 602 may also include an interface 605 (e.g. screen, monitor, graphical user interface, audio device interface, etc.) that may enable the first user 601 to interact with various applications executing on the first user device 602, to interact with various applications executing within the system 600, and to interact with the system 600 itself In certain embodiments, the first user device 602 may be a computer, a laptop, a tablet device, a phablet, a server, a mobile device, a smartphone, a smart watch, and/or any other type of computing device. Illustratively, the first user device 602 is shown as a mobile device in FIG. 11. The first user device 602 may also include a global positioning system (GPS), which may include a GPS receiver and any other necessary components for enabling GPS functionality, accelerometers, gyroscopes, sensors, and any other componentry suitable for a mobile device. In certain embodiments, the first user device 602 may be configured to include any number of sensors, such as, but not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof In certain embodiments, the first user device 602 may be configured to communicate with any of the components of the garment sleeve 110 and/or any of the components of FIGS. 1-13.

In addition to the first user 601, the system 600 may include a second user 610, who may utilize a second user device 611 to access data, content, and applications, or to perform a variety of other tasks and functions. As with the first user 601, the second user 610 may be a user that is seeking to monitor herself. However, in certain embodiments, the second user 610 may be a health professional or other individual that is monitoring the first user 601. Much like the first user 601, the second user 610 may utilize second user device 611 to access an application (e.g. a browser or a mobile application) executing on the second user device 611 that may be utilized to access web pages, data, and content associated with the system 600. The second user device 611 may include a memory 612 that includes instructions, and a processor 613 that executes the instructions from the memory 612 to perform the various operations that are performed by the second user device 611. In certain embodiments, the processor 613 may be hardware, software, or a combination thereof The second user device 611 may also include an interface 614 (e.g. a screen, a monitor, a graphical user interface, etc.) that may enable the second user 610 to interact with various applications executing on the second user device 611, to interact with various applications executing in the system 600, and to interact with the system 600. In certain embodiments, the second user device 611 may be a computer, a laptop, a tablet device, a phablet, a server, a mobile device, a smartphone, a smart watch, and/or any other type of computing device. Illustratively, the second user device 611 may be a computing device in FIG. 11. The second user device 611 may also include any of the componentry described for first user device 602. The second user device 611 may similarly be configured to communicate with any of the components of the garment sleeve 100 and/or any of the components of FIGS. 1-13.

In certain embodiments, the first user device 602 and the second user device 611 may have any number of software applications and/or application services stored and/or accessible thereon. For example, the first and second user devices 602, 611 may include applications for measuring and analyzing biometric data, applications for determining and analyzing conditions associated with monitored objects and/or physical structures, applications for analyzing sensor data, applications for determining and analyzing health conditions, applications for determining and analyzing the physiological status of a user, applications for generating alerts, applications for analyzing and interpreting sensor data, artificial intelligence applications, machine learning applications, big data applications, applications for analyzing data, applications for integrating data, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 601, 610 to readily interact with the software applications.

The software applications and services may also be utilized by the first and second users 601, 610 to interact with any device in the system 600, any components of the garment sleeve 100, any network in the system 600, or any combination thereof. For example, the software applications executing on the first and second user devices 602, 611 may be applications for receiving data, applications for measuring and analyzing biometric data, applications for monitoring physical structures and/or bodies, applications for storing data, applications for analyzing sensor data, applications for determining health conditions, applications for determining how to respond to a health condition, applications for determining a physiological status of a user, applications for determining how to respond to an environmental condition (e.g. an environmental condition that may affect the first user 601), applications for receiving demographic and preference information, applications for transforming data, applications for executing mathematical algorithms, applications for generating and transmitting electronic messages, applications for generating and transmitting various types of content, any other type of applications, or a combination thereof In certain embodiments, the first and second user devices 602, 611 may include associated telephone numbers, internet protocol addresses, device identities, or any other identifiers to uniquely identify the first and second user devices 602, 611 and/or the first and second users 601, 610. In certain embodiments, location information corresponding to the first and second user devices 602, 611 may be obtained based on the internet protocol addresses, by receiving a signal from the first and second user devices 602, 611, or based on profile information corresponding to the first and second user devices 602, 611.

The system 600 may also include a communications network 635. The communications network 635 of the system 600 may be configured to link each of the devices in the system 600 to one another. For example, the communications network 635 may be utilized by the first user device 602 to connect with other devices within or outside communications network 635. Additionally, the communications network 635 may be configured to transmit, generate, and receive any information and data traversing the system 600. In certain embodiments, the communications network 635 may include any number of servers, databases, or other componentry, and may be controlled by a service provider. The communications network 635 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof Illustratively, server 640 and server 650 are shown as being included within communications network 635. The communications network 635 may also be utilized to link each of the components and devices of the garment sleeve 100 and/or any other components of FIGS. 1-13 to each other and/or to the system 600.

Notably, the functionality of the system 600 may be supported and executed by using any combination of the servers 640, 650, and 660. The servers 640, and 650 may reside in communications network 635, however, in certain embodiments, the servers 640, 650 may reside outside communications network 635. The servers 640, and 650 may be utilized to perform the various operations and functions provided by the system 600, such as those requested by applications executing on the first and second user devices 602, 611. In certain embodiments, the server 640 may include a memory 641 that includes instructions, and a processor 642 that executes the instructions from the memory 641 to perform various operations that are performed by the server 640. The processor 642 may be hardware, software, or a combination thereof. Similarly, the server 650 may include a memory 651 that includes instructions, and a processor 652 that executes the instructions from the memory 651 to perform the various operations that are performed by the server 650. In certain embodiments, the servers 640, 650, and 660 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 640, 650 may be communicatively linked to the communications network 635, any network, any device in the system 600, or any combination thereof.

The database 655 of the system 600 may be utilized to store and relay information that traverses the system 600, cache information and/or content that traverses the system 600, store data about each of the devices in the system 600, and perform any other typical functions of a database. In certain embodiments, the database 655 may store the output from any operation performed by garment sleeve 100 and/or computing system 510, operations performed and/or outputted by any component, program, process, device, network of the system 600, or any combination thereof. For example, the database 655 may store data from data sources, such as, but not limited to, garment sleeve 100, computing system 510, or a combination thereof The database 655 may store information relating to the monitored electrical resistances values, electricity passing through the sensors, changes in voltage, changes in conduction, any electrical or other properties, and any physical properties and/or information monitored by the garment sleeve 100, the computing system 510, and/or the system 600. In certain embodiments, the database 655 may be connected to or reside within the communications network 635, any other network, or a combination thereof. In certain embodiments, the database 655 may serve as a central repository for any information associated with any of the devices and information associated with the system 600 and/or garment sleeve 100. Furthermore, the database 655 may include a processor and memory or be connected to a processor and memory to perform the various operations associated with the database 655. In certain embodiments, the database 655 may be connected to the servers 640, 650, 660, the first user device 602, the second user device 611, any devices in the system 600, the garment sleeve 100, the computing system 510, any other device, any network, or any combination thereof.

The database 655 may also store information obtained from the system 600, store information associated with the first and second users 601, 610, store location information for the first and second user devices 602, 611 and/or first and second users 601, 610, store user profiles associated with the first and second users 601, 610, store device profiles associated with any device in the system 600, the garment sleeve 100 and/or computing system 510, store communications traversing the system 600, store user preferences, store demographic information for the first and second users 601, 610, store information associated with any device or signal in the system 600, store information relating to usage of applications accessed by the first and second user devices 602, 611, store any information obtained from any of the networks in the system 600, store historical data associated with the first and second users 601, 610, store device characteristics, store information relating to any devices associated with the first and second users 601, 610, or any combination thereof The database 655 may store algorithms for analyzing sensor data obtained from the garment sleeve 100, algorithms for determining events, such as health conditions and/or physiological status, algorithms conducting artificial intelligence and/or machine learning, algorithms for comparing sensor data to baseline and/or threshold values, any other algorithms for performing any other calculations and/or operations in the system 600, or any combination thereof. The database 655 may also be configured to store information relating to detected conditions and/or events, actions to perform in response to the detected conditions and/or events, information indicating whether one or more of the actions have been performed, any other information provided by the system 600 and/or method 700, or any combination thereof In certain embodiments, the database 655 may be configured to store any information generated and/or processed by the system 600, store any of the information disclosed for any of the operations and functions disclosed for the system 600 herewith, store any information traversing the system 600, or any combination thereof. Furthermore, the database 655 may be configured to process queries sent to it by any device in the system 600, the garment sleeve 100, and/or computing system 510.

The system 600 may also include an external network 665. The external network 665 of the system 600 may be configured to link each of the devices in the system 600 to one another. For example, the external network 665 may be utilized by the first user device 602, the second user device 611, the garment sleeve 100, and/or the computing system 510 to connect with other devices within or outside communications network 635. Additionally, the external network 665 may be configured to transmit, generate, and receive any information and data traversing the system 600. In certain embodiments, the external network 665 may include any number of servers, databases, or other componentry, and may be controlled by a service provider. The external network 665 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. In certain embodiments, the external network 665 may be outside the system 600 and may be configured to perform various functionality provided by the system 600, such as if the system 600 is overloaded and/or needs additional processing resources.

Notably, as shown in FIG. 11, the system 600 may perform any of the operative functions disclosed herein by utilizing the processing capabilities of server 660, the storage capacity of the database 655, or any other component of the system 600 to perform the operative functions disclosed herein. The server 660 may include one or more processors 662 that may be configured to process any of the various functions of the system 600. The processors 662 may be software, hardware, or a combination of hardware and software. Additionally, the server 660 may also include a memory 661, which stores instructions that the processors 662 may execute to perform various operations of the system 600. For example, the server 660 may assist in processing loads handled by the various devices in the system 600, such as, but not limited to, positioning the fabric onto a body part of a user; assessing a resistance of a portion of conductive elastic material in the fabric; assessing motion of a first portion of a body part using a first inertial measurement unit; assessing motion of a second portion of a body part using a second inertial measurement unit; receiving resistance data and motion data from the first and second inertial measurement units; assessing a shape, a position and/or a movement of the body part based on the resistance data and motion data; transmitting output signals relating to the assessed shape, position, and/or movement of the user; and performing any other suitable operations conducted in the system 600 or otherwise. In one embodiment, multiple servers 660 may be utilized to process the functions of the system 600. The server 660 and other devices in the system 600, may utilize the database 655 for storing data about the devices in the system 600 or any other information that is associated with the system 600. In one embodiment, multiple databases 655 may be utilized to store data in the system 600.

In certain embodiments, the system 600 may also include a computing device 670. The computing device 670 may include one or more processors 672 that may be configured to process any of the various functions of the system 600. The processors 672 may be software, hardware, or a combination of hardware and software. Additionally, the computing device 670 may also include a memory 671, which stores instructions that the processors 672 may execute to perform various operations of the system 600. For example, the computing device 670 may assist in processing loads handled by the various devices in the system 600, such as, but not limited to, devices and components of the garment sleeve 100 and/or computing system 510.

Although the figures illustrate specific example configurations of the various components of the system 600, the system 600 may include any configuration of the components, which may include using a greater or lesser number of the components. For example, the system 600 is illustratively shown as including a first user device 602, a second user device 611, a communications network 635, a server 640, a server 650, a server 660, a database 655, and an external network 665. However, the system 600 may include multiple first user devices 602, multiple second user devices 611, multiple databases 625, multiple communications networks 635, multiple servers 640, multiple servers 650, multiple servers 660, multiple databases 655, multiple external networks 665, and/or any number of any of the other components inside or outside the system 600. Similarly, the system 600 may include any number of data sources, applications, systems, and/or programs. Notably, any of the components of the system 600 may be integrated into and/or communicatively coupled to the garment sleeve 100 and/or computing system 510. Furthermore, in certain embodiments, substantial portions of the functionality and operations of the system 600 may be performed by other networks and systems that may be connected to system 600.

In embodiments described herein, any of the electronic components of sleeve 100 may include a waterproof coating (e.g., a waterproof nanocoating) adhered to the exterior of the electronic components. The coating may allow for the components to function properly when sleeve 100 is exposed to a wet environment that may include sweat and/or water.

In embodiments described herein, wiring connecting two or more electronic components found in sleeve 100 may be contained within a multi-layered fabric construction. In some embodiments, the wiring may be partially engrained within seams in sleeve 100. In some embodiments, the wiring may comprise conductive fibers. The conductive fibers may be in the form of one or more yarns woven or knit with other fibers. In some embodiments, the yarns may be coated with an insulative polymer to, for example, provide efficient transfer of power or data.

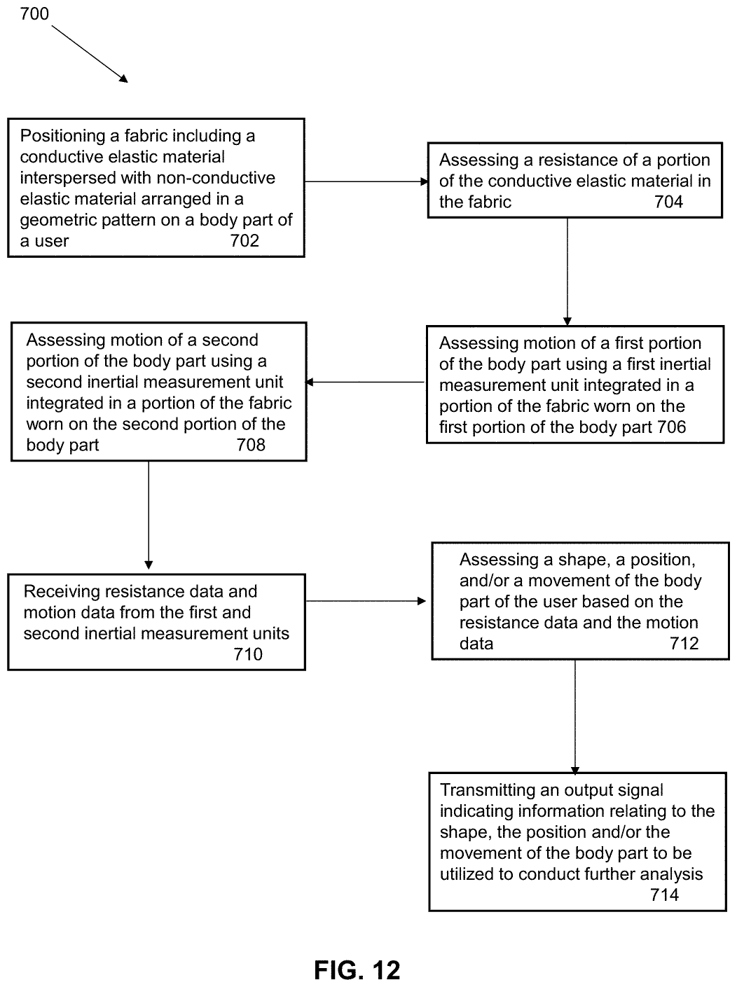

As shown in FIG. 12, an exemplary method 700 for conducting biometric monitoring using a garment sleeve 100 is schematically illustrated. The method 700 may include, at step 702, positioning a fabric of a garment sleeve 100 including a conductive elastic material interspersed with non-conductive elastic material arrange in a geometric pattern on a body part of a user. In certain embodiments, the positioning may be performed and/or facilitated by utilizing any of the components of the garment sleeve 100, the computing system 510, any of the components of system 600, any other components, programs, devices, and/or individuals, or a combination thereof. At step 704, the method 700 may include assessing a resistance of a portion of the conductive elastic material in the fabric of the garment sleeve 100. In certain embodiments, the assessing may be performed and/or facilitated by utilizing any of the components of the garment sleeve 100, the computing system 510, any of the components of system 600, any other components, programs, devices, and/or individuals, or a combination thereof

At step 706, the method 700 may include assessing motion of a first portion of the body part using a first inertial measurement unit integrated in a portion of the fabric worn on the first portion of the body part. In certain embodiments, the assessing may be performed and/or facilitated by utilizing any of the components of the garment sleeve 100, the computing system 510, any of the components of system 600, any other components, programs, devices, and/or individuals, or a combination thereof. At step 708, the method 700 may include assessing motion of a second portion of the body part using a second inertial measurement unit integrated in a portion of the fabric worn on the second portion of the body part. In certain embodiments, the assessing may be performed and/or facilitated by utilizing any of the components of the garment sleeve 100, the computing system 510, any of the components of system 600, any other components, programs, devices, and/or individuals, or a combination thereof.

At step 710, the method 700 may include receiving resistance data and motion data from the first and second inertial measurement units and/or other components of the garment sleeve 100. In certain embodiments, the receiving may be performed and/or facilitated by utilizing any of the components of the garment sleeve 100, the computing system 510, any of the components of system 600, any other components, programs, devices, and/or individuals, or a combination thereof. At step 712, the method 700 may include assessing a shape, a position, and/or a movement of the body part of the user based on the resistance data and/or the motion data. In certain embodiments, the assessing may be performed and/or facilitated by utilizing any of the components of the garment sleeve 100, the computing system 510, any of the components of system 600, any other components, programs, devices, and/or individuals, or a combination thereof At step 714, the method 700 may include transmitting an output signal indicating information relating to the shape, the position and/or the movement of the body part to be utilized to conduct further analysis. For example, the further analysis may be utilized to determine a health condition of the user, an ailment of the user, a strength of the user, a physical capability of the user, any biometric information about the user, any other information, or a combination thereof. In certain embodiments, the transmitting of the signal may be performed and/or facilitated by utilizing any of the components of the garment sleeve 100, the computing system 510, any of the components of system 600, any other components, programs, devices, and/or individuals, or a combination thereof.

Referring now also to FIG. 8, at least a portion of the methodologies and techniques described with respect to the exemplary embodiments of the system 600, the garment sleeve 100, and/or the computing system 510 can incorporate a machine, such as, but not limited to, computer system 800, or other computing device within which a set of instructions, when executed, may cause the machine to perform any one or more of the methodologies or functions discussed above. The machine may be configured to facilitate various operations conducted by the system 600, the garment sleeve 100, and/or the computing system 510. For example, the machine may be configured to, but is not limited to, assist the system 600 by providing processing power to assist with processing loads experienced in the system 600, by providing storage capacity for storing instructions or data traversing the system 600, or by assisting with any other operations conducted by or within the system 600.

In some embodiments, the machine may operate as a standalone device. In some embodiments, the machine may be connected (e.g., using communications network 635, another network, or a combination thereof) to and assist with operations performed by other machines, programs, functions, and systems, such as, but not limited to, the first user device 602, the second user device 611, the server 640, the server 650, the database 655, the server 660, the external network 665, the communications network 635, the garment sleeve 100, the computing system 510, any device, system, and/or program, or any combination thereof. The machine may be connected with any component in the system 600. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 800 may include a processor 802 (e.g., a central processing unit (CPU), a graphics processing unit (GPU, or both), a main memory 804 and a static memory 806, which communicate with each other via a bus 808. The computer system 800 may further include a video display unit 810, which may be, but is not limited to, a liquid crystal display (LCD), a flat panel, a solid state display, or a cathode ray tube (CRT). The computer system 800 may include an input device 812, such as, but not limited to, a keyboard, a cursor control device 814, such as, but not limited to, a mouse, a disk drive unit 816, a signal generation device 818, such as, but not limited to, a speaker or remote control, and a network interface device 820.

The disk drive unit 816 may include a machine-readable medium 822 on which is stored one or more sets of instructions 824, such as, but not limited to, software embodying any one or more of the methodologies or functions described herein, including those methods illustrated above. The instructions 824 may also reside, completely or at least partially, within the main memory 804, the static memory 806, or within the processor 802, or a combination thereof, during execution thereof by the computer system 800. The main memory 804 and the processor 802 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 822 containing instructions 824 so that a device connected to the communications network 635, the external network 665, another network, or a combination thereof, can send or receive voice, video or data, and communicate over the communications network 635, the external network 465, another network, or a combination thereof, using the instructions. The instructions 624 may further be transmitted or received over the communications network 635, the external network 665, another network, or a combination thereof, via the network interface device 820.

While the machine-readable medium 822 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

What is claimed is:

1. A system, comprising:
a fabric for being worn on a limb of a wearer, wherein the fabric comprises one or more layers, wherein a layer of the one or more layers of the fabric comprises a geometric pattern of conductive elastic material interspersed with non-conductive elastic material;
a plurality of nodes integrated in the fabric, wherein a first portion of the conductive elastic material is coupled between at least two nodes;
a positioning component integrated in the fabric;
a first inertial measurement unit integrated in the fabric and positioned on a first side of the positioning component;
a second inertial measurement unit integrated in the fabric and positioned on a second side of the positioning component; and
a processor integrated in the fabric, wherein the processor is configured to assess a resistance of the first portion of the conductive elastic material between the at least two nodes and receive data from the first and second inertial measurement units.

2. The system of claim 1, wherein the processor is configured to assess a shape, a position, and a movement of the limb using the data received.

3. The system of claim 2, wherein the processor is configured to provide an output indicative of the shape, the position, and the movement of the limb.

4. The system of claim 1, wherein the positioning component is configured to be positioned at a joint of the limb when the fabric is worn on the limb.

5. The system of claim 1, wherein a sensor is positioned at one of the nodes.

6. The system of claim 5, wherein the processor is electrically coupled to the sensor and the first inertial measurement unit, the second inertial measurement unit, or a combination thereof

7. The system of claim 1, wherein the at least two nodes are positioned at corners of a geometric in the geometric pattern.

8. The system of claim 1, wherein a third node is coupled to at least one of the at least two nodes with a second portion of the conductive elastic material.

9. The system of claim 1, wherein the first inertial measurement unit, the second inertial measurement unit, or a combination thereof, is configured to assess a physical position of the limb of the wearer in a three-dimensional space.

10. The system of claim 1, further comprising a heart rate monitor integrated in the fabric.

11. The system of claim 1, further comprising a galvanic skin response sensor integrated in the fabric.

12. The system of claim 1, further comprising an oxygen saturation monitor integrated in the fabric.

13. The system of claim 4, wherein the first inertial measurement unit is positioned above the joint of the limb when the fabric is worn on the limb, and wherein the second inertial measurement unit is positioned below the joint of the limb when the fabric is worn on the limb.

14. The system of claim 1, wherein the layer of the fabric comprising the conductive elastic material is configured to be in contact with the skin of the wearer of the fabric.

15. A method, comprising:
assessing a resistance of a portion of a conductive elastic material interspersed with non-conductive elastic material in a geometric pattern in a fabric, wherein the fabric is worn on a limb of a user;
assessing motion of a first portion of the limb using a first inertial measurement unit integrated in a portion of the fabric worn on the first portion of the limb;
assessing motion of a second portion of the limb using a second inertial measurement unit integrated in a portion of the fabric worn on the second portion of the limb;
receiving, in a processor integrated in the fabric, resistance data and motion data from the first inertial measurement unit and the second inertial measurement unit; and
assessing a shape, a position, and a movement of the limb of the user using the resistance data and the motion data.

16. The method of claim 15, further comprising providing, from the processor, an output indicative of the shape, the position, and the movement of the limb.

17. The method of claim 15, further comprising transmitting, from the processor, a wireless output signal comprising data for the shape, the position, and the movement of the limb.

18. The method of claim 15, wherein the position and the movement of the limb are assessed based on the motions of the first and second portions of the limb assessed by the first inertial measurement unit and the second inertial measurement unit.

19. The method of claim 15, wherein assessing the shape, the position, and the movement of the limb comprises assessing the shape, the position, and the movement of the limb in a three-dimensional space.

20. The method of claim 15, further comprising assessing a heart rate of the wearer using a heart rate monitor integrated in the fabric.

21. The method of claim 15, further comprising assessing the motion of the limb of the wearer in nine degrees of freedom using the first inertial measurement unit.
